(19) United States
(12) Patent Application Publication
(10) Pub. No.: US 2026/0306042 A1
(43) Pub. Date: Oct. 8, 2026

(54) Dynamic Frame Rate Adjustments in Digital Subtraction Angiography

(71) Applicant: UNIVERSITY OF UTAH RESEARCH FOUNDATION, Salt Lake City, UT (US)

(72) Inventors: Brendan T. CRABB, SALT LAKE CITY, UT (US); Gabriel C. FINE, SALT LAKE CITY, UT (US); Frederic NOO, SALT LAKE CITY, UT (US)

(21) Appl. No.: 19/481,756

(22) PCT Filed: May 3, 2024
(86) PCT No.: PCT/US2024/027874

§ 371 Date: Nov. 5, 2025

Related U.S. Application Data
(60) Provisional application No. 63/464,286, filed on May 5, 2023.

(51) Int. Cl. A61B 6/00; A61B 6/50; G06T 5/50; G06T 7/20

(52) U.S. Cl. CPC A61B 6/5264; A61B 6/504; G06T 5/50; G06T 7/20; G06T 2207/10116; G06T 2207/20072; G06T 2207/20081; G06T 2207/20224; G06T 2207/30016; G06T 2207/30061; G06T 2207/30101

(57) ABSTRACT

Disclosed is a method for obtaining digital subtraction angiography (DSA) images using a dynamic frame rate. The method dynamically adjusts frame rates in response to motion artifacts during DSA image acquisition. In situations where there is a high degree of motion, the frame rate can be optimized to increase image quality. On the other hand, in situations where there is a low degree of motion, the frame rate can be lowered to limit the radiation exposure to the patient and provider without overly sacrificing image quality.

### CROSS-REFERENCE TO RELATED APPLICATIONS

This application claims priority to and the benefit of U.S. Provisional Application No. 63/464,286, filed May 5, 2023, and titled “Dynamic Frame Rate Adjustments in Digital Subtraction Angiography,” the entirety of which is incorporated herein by reference.

### BACKGROUND

Digital subtraction angiography (DSA) is a commonly used method for visualizing vasculature throughout the human body. Typically, X-ray images are taken before and after injection of a contrast agent. The pre-injection image (referred to as the mask image or mask frame) provides a baseline of the target area under examination. The contrast agent, often an iodine-based contrast agent, absorbs the X-ray radiation and appears relatively bright on the post-injection images. The mask images are digitally subtracted from the post-injection images. This process eliminates other tissues from the images, leaving only the contrast-filled blood vessels visible. The resulting images provide a clearer view of the blood vessels without the surrounding structures.

Unfortunately, patient motion can generate artifacts, limiting the diagnostic quality of the images. When motion artifacts are present, image quality can be effectively improved by utilizing higher acquisition frame rates. However, higher frame rates increase the radiation dose to the patient and potentially also to the provider. Moreover, frame rate adjustments are currently performed manually, with ad-hoc approaches that are not reproducible, not automated, and which can differ from physician to physician.

Accordingly, there is an ongoing need for approaches to DSA that can improve image quality and/or reduce radiation exposure to patients and physicians.

### SUMMARY

The present disclosure relates to a method to dynamically adjust frame rates on a series-to-series (i.e., image set to image set) basis in response to motion artifacts during DSA image acquisition. For example, in situations where there is a high degree of motion, the frame rate can be optimized to increase image quality. On the other hand, in situations where there is a low degree of motion, the frame rate can be lowered to limit the radiation exposure to the patient and provider without overly sacrificing image quality. The disclosed approach can both improve image quality when patient motion is present, while also limiting radiation exposure when high frame rates are not necessary.

In one embodiment, a method of generating digital DSA images using a dynamic frame rate comprises selecting an initial imaging frame rate for X-ray imaging of target anatomy; obtaining a mask image of the target anatomy; obtaining a first set of images at the initial imaging frame rate; generating a first set of DSA images from the first set of images via digital subtraction of the mask image; determining a degree of motion in the first set of DSA images; adjusting the initial imaging frame rate to a first adjusted frame rate based on the determined degree of motion in the first set of DSA images; obtaining a second set of images at the first adjusted frame rate; and generating a second set of DSA images from the second set of images via digital subtraction of the mask image.

The method can further comprise determining a degree of motion for multiple successive sets of DSA images and adjusting the frame rate for each subsequent set of DSA images based on the most recent degree of motion measured.

The degree of motion in a set of DSA images can be determined by: generating an image histogram from at least one DSA image of the first set of DSA images; calculating a standard deviation of pixel intensities from the image histogram; and determining the degree of motion in the first set of DSA images based on the calculated standard deviation of pixel intensities, wherein the degree of motion is positively correlated to the standard deviation of pixel intensities. The degree of motion can additionally or alternatively be determined utilizing a machine learning algorithm, wherein the machine learning algorithm is generated using a machine learning process in which DSA images pre-categorized according to degree of motion are used as a training dataset.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the disclosure will become apparent and more readily appreciated from the following description, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1A-1C illustrate the general principles of DSA, with FIG. 1A showing a mask image captured prior to injection of contrast agent, FIG. 1B showing a native image taken after injection of the contrast agent, and FIG. 1C showing the resulting DSA image after the mask image of FIG. 1A is digitally subtracted from the post-injection native image of FIG. 1B;

FIG. 2 is an example of a DSA image with obscured features due to motion artifacts resulting from patient motion;

FIG. 3 illustrates the concept of frame rate in the context of DSA;

FIG. 4 is a flow chart of an example method for generating DSA images using a dynamic frame rate;

FIGS. 5A-5D illustrate representative examples of DSA images and corresponding pixel intensity histograms, where FIG. 5A is an example of a DSA image with a relatively low level of motion artifacts and FIG. 5B is its corresponding histogram, and FIG. 5C is an example of a DSA image with a relatively high level of motion artifacts and FIG. 5D is its corresponding histogram; and

FIGS. 6A and 6B illustrate boxplots and categorical scatterplots, respectively, showing a strong association between the presence of motion artifacts and the standard deviation of pixel intensities.

### DETAILED DESCRIPTION

### Overview of Digital Subtraction Angiography

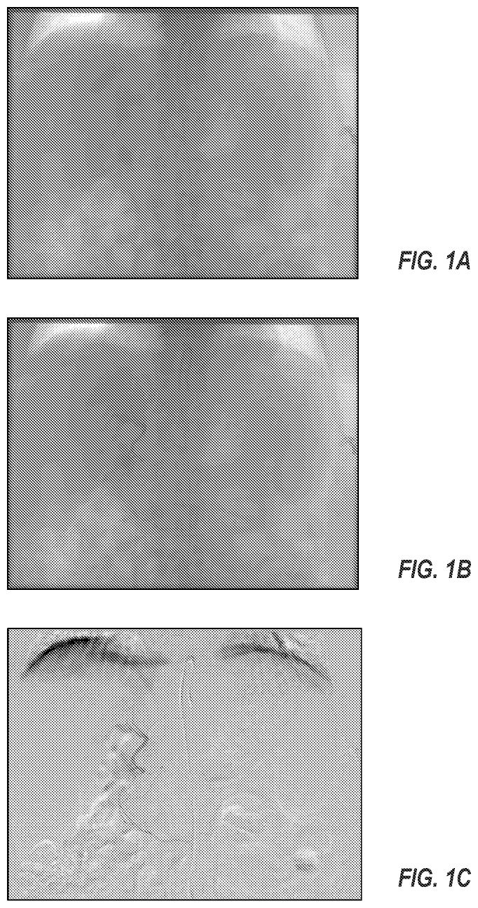

FIGS. 1A-1C illustrate the general principles of DSA. FIG. 1A shows a mask image captured prior to injection of contrast agent. The target vasculature to be imaged is not visible. FIG. 1B shows a native image taken after injection of the contrast agent. Although the target vasculature is somewhat more visible relative to the pre-contrast image of FIG. 1A, the vasculature is still rather obscured by the surrounding tissues. FIG. 1C shows the resulting DSA image after the mask image of FIG. 1A is digitally subtracted from the post-injection native image of FIG. 1B.

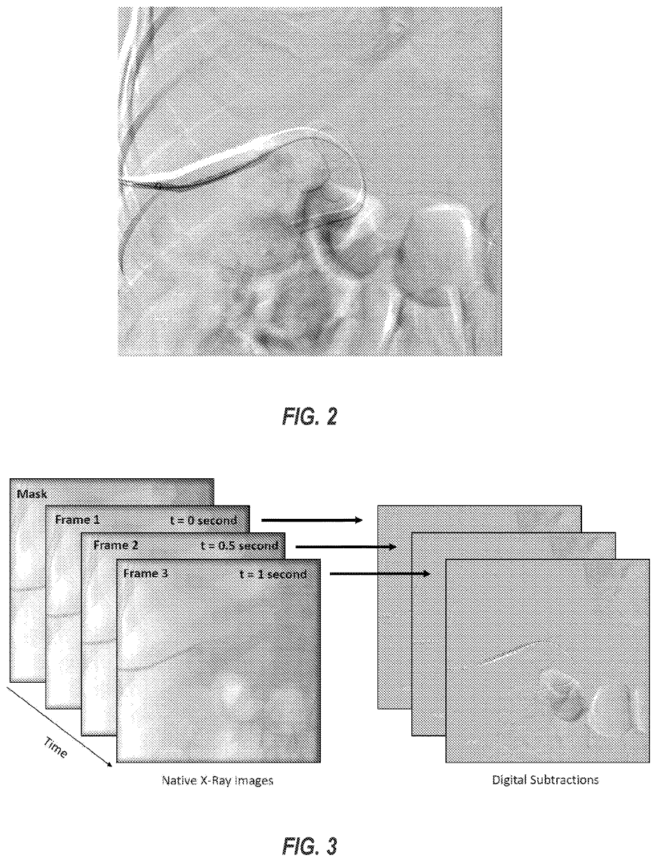

FIG. 2 is an example of a DSA image with obscured features due to motion artifacts resulting from patient motion. That is, patient motion during image acquisition creates misalignment between the subtracted images, resulting in visible motion artifacts. FIG. 3 illustrates the concept of frame rate in the context of DSA. The number of frames captured and generated over time is often reported as frames per second (fps). Motion artifacts can be minimized with higher frame rates. However, higher frame rates necessarily mean the patient and possibly the practitioner are exposed to higher levels of X-ray radiation.

Radiologists typically refer to static X-ray images as “radiographs.” Fluoroscopy employs a continuous X-ray beam to create real-time moving images of the internal structures of a patient. In DSA, fluoroscopy is used to capture the dynamic movement of the contrast agent through the target vasculature. The resulting images are thus often referred to as “fluoroscopic images.” As used herein, general terms such as “X-ray images,” when used in the context of a DSA process, refer to dynamic images captured during DSA and may therefore also be appropriately referred to as fluoroscopic images. It will also be understood, however, that lay terminology often ignores or misunderstands this distinction. Accordingly, subject matter should not be considered as outside the scope of this application simply due to the use of lay terminology.

### Dynamic Frame Rate Adjustment

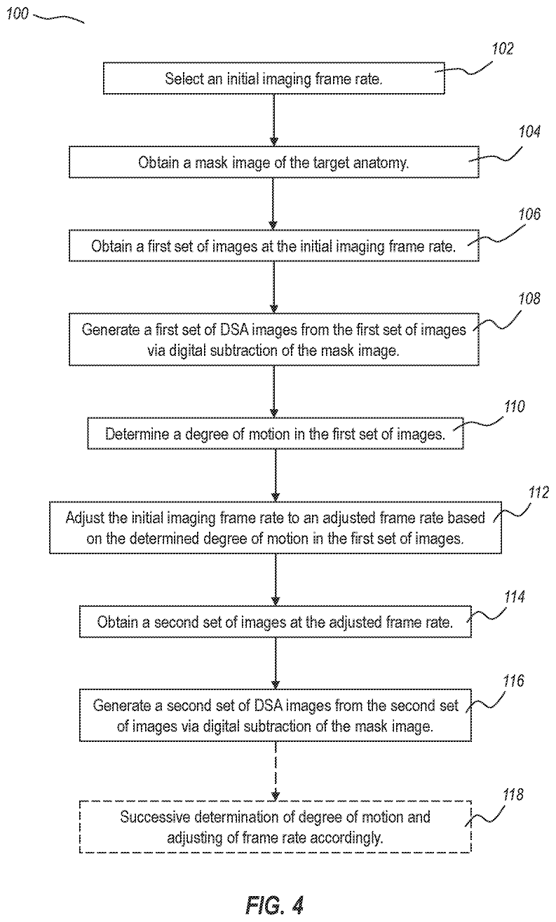

FIG. 4 is a flow chart of an example method 100 for generating DSA images using a dynamic frame rate. The method can include the step of selecting an initial imaging frame rate for X-ray imaging of target anatomy (step 102). The initial imaging frame rate can be selected based on conventional frame rate selection criteria. For example, conventional approaches typically select frame rate ranges based on the type of procedure to be carried out. That is, the initial imaging frame rate can be selected based on the targeted anatomy for imaging. Example initial imaging frame rates include 1-3 fps for cerebral angiography, 4-12 fps for visceral angiography, and 15-30 fps for pulmonary angiography. These are exemplary only and practitioners can select different initial imaging frame rates according to particular application needs, specific patient requirements, and/or specific procedural circumstances.

The method 100 also includes the steps of obtaining a mask image of the target anatomy (step 104), obtaining a first set of images at the initial imaging frame rate (step 106), and generating a first set of DSA images from the first set of images via digital subtraction of the mask image (step 108). These steps can be carried out using DSA equipment and procedures as known in the art. The target anatomy is the anatomical region that includes the vasculature of interest intended to be imaged during the DSA procedure.

The method 100 also includes the step of determining a degree of motion in the first set of DSA images (step 110). Different approaches for accomplishing this step are described in more detail below. In some embodiments, the degree of motion in the first set of DSA images is positively correlated to the standard deviation of pixel intensities from one or more of the image histograms corresponding to the images within the first set of DSA images. Other embodiments can additionally or alternatively utilize a machine learning algorithm, a deep-learning approach, and/or a computer vision approach.

The degree of motion can be determined according to any suitable form. For example, the degree of motion can be calculated as a value within a numerical scale (e.g., 0 to 10, 1 to 5, 0 to 100, or any other desired numerical scale), or as a value within a Likert scale (e.g., a low/medium/high scale, a five-point Likert scale, or any other desired Likert scale). The granularity of the degree of motion scale can be selected to a desired level based on practitioner preference, patient type, procedure type, and the like. For example, a simple low/medium/high scale may be sufficient for many applications, though a more granular numerical scale may be desired in other applications.

The method 100 also includes the step of adjusting the initial imaging frame rate to an adjusted frame rate based on the determined degree of motion in the first set of DSA images (step 112). The frame rate adjustment can depend on how well the initial imaging frame rate “matches” the determined degree of motion. For example, a desired frame rate scale can be overlayed with the degree of motion scale such that each value on the degree of motion scale (or subrange of such values) corresponds to a desired frame rate (or subrange of frame rates). The frame rate can be adjusted to align to the corresponding degree of motion according to the overlying scales.

The particular way in which the degree of motion scale and the frame rate scale correspond to one other can be varied. For example, frame rates of about 15-30 fps can be considered “high”, frame rates of about 4-12 fps can be considered “medium”, and frame rates of about 1-3 fps can be considered “low”, though these correlations can be different in certain applications and/or can be customized according to practitioner preferences, patient type, procedure type, and the like. Similarly, more granular degree of motion scales will have more granular corresponding frame rate ranges.

If the initial imaging frame rate substantially matches the corresponding portion of the degree of motion scale, the frame rate can be determined as appropriate for the given degree of motion. If, however, a measured degree of motion is higher or lower than the corresponding frame rate, the frame rate can be raised or lowered accordingly. In some embodiments, adjusting the initial frame rate comprises increasing the frame rate when the determined degree of motion of the first set of DSA images surpasses a threshold and comprises decreasing the frame rate when the determined degree of motion of the first set of DSA images is below a threshold.

The threshold values can be set values or more preferably can be sliding values that depend upon the initial imaging frame rate. That is, if the initial imaging frame rate is relatively high, the motion thresholds for raising or lowering the frame rate will be correspondingly higher, and if the initial imaging frame rate is relatively low, the motion thresholds for raising or lowering the frame rate will be correspondingly lower.

The method 100 can also include the steps of obtaining a second set of images at the adjusted frame rate (step 114) and generating a second set of DSA images from the second set of images via digital subtraction of the mask image (step 116). In other words, an additional set of DSA images can be obtained at the updated, adjusted frame rate. The adjusted frame rate can better align the amount of radiation delivered to the patient to the amount of measured motion to optimize the balance between image quality and the desire to minimize radiation exposure.

The method can further include successive determination of the degree of motion in successive sets of DSA images and adjusting of frame rate accordingly (step 118). That is, the degree of motion can be iteratively determined, and the frame rate can be iteratively adjusted, between successive sets of images to continuously update the frame rate according to the determined degree of motion. For example, the method 100 can continue by determining a degree of motion for each of multiple successive sets of DSA images and adjusting the frame rate for each subsequent set of DSA images based on the most recent degree of motion measured. In this manner, the frame rate can be adjusted with a sufficient frequency to minimize radiation exposure without sacrificing DSA image quality. The number of images within each set of images can be modified so that motion is measured frequently enough, and the frame rate is adjusted frequently enough, to achieve desired alignment between frame rate and patient motion. This can vary depending on procedure, patient, equipment, and the like.

The disclosed approach can be carried out using a system designed to operate in conjunction with conventional DSA equipment. For example, a computer system can be communicatively coupled to an X-ray device and can be configured to control the X-ray device to carry out the disclosed approach. The computer system can be configured to: receive input (e.g., user input) selecting an initial imaging frame rate for X-ray imaging of target anatomy; instruct the X-ray device to obtain a mask image of the target anatomy; instruct the X-ray device to obtain a first set of images at the initial imaging frame rate; generate a first set of DSA images from the first set of images via digital subtraction of the mask image; determine a degree of motion in the first set of DSA images; adjust the initial imaging frame rate to a first adjusted frame rate based on the determined degree of motion in the first set of DSA images; instruct the X-ray device to obtain a second set of images at the first adjusted frame rate; and generate a second set of DSA images from the second set of images via digital subtraction of the mask image. As discussed above, the system can continue to measure a degree of motion in successive DSA image sets and can continuously adjust the frame rate accordingly.

A paper from Haouchine et al. discloses a method that uses machine learning to simulate high frame rate acquisitions from low frame rate inputs. The authors state that this method generates synthetic acquisitions with fewer artifacts. (Haouchine, Nazim, et al. “Estimation of High Framerate Digital Subtraction Angiography Sequences at Low Radiation Dose.” Medical Image Computing and Computer Assisted Intervention—MICCAI 2021:24th International Conference, Strasbourg, France, Sep. 27-Oct. 1, 2021, Proceedings, Part VI 24. Springer International Publishing, 2021).

However, the Haouchine et al. method and similar methods rely on post-processing steps that do not change the actual radiation dose delivered to the patient. In contrast, the presently disclosed solution can directly adjust the acquisition frame rate and consequently the radiation dose being delivered to the patient.

Moreover, the Haochine et al. method and similar methods are unidirectional. In some scenarios, high frame rates may be necessary despite postprocessing attempts. The disclosed method is bidirectional and can both increase and decrease the frame rate at different stages during DSA image acquisition, depending on determined optimization.

Further, the Haochine et al. method, and similar methods, rely on the generation of artificial images. Artificial images generated from machine learning techniques are prone to incorporating artifacts and/or irregularities. In contrast, the presently disclosed approach improves the quality of real images by dynamically adjusting and updating the frame rate in an intelligent manner.

### Assessing the Degree of Motion

As discussed above, the degree of motion of a DSA image set can be determined in a variety of ways. In one embodiment, determining the degree of motion includes generating an image histogram from at least one DSA image of the previous (e.g., immediately prior) set of DSA images. An image histogram measures the distribution of pixel intensity values in an image. In a grayscale image such as used for DSA imaging, pixel intensity typically ranges from 0 (black) to 255 (white), with shades of gray in between. An image histogram can be graphically represented with the X-axis representing the range of pixel intensity values and the Y-axis representing the frequency/count of pixels that have the particular pixel intensity value. As used herein, generating an image histogram refers to generating the underlying data, and the relevant embodiments need not actually generate a graphical histogram.

The standard deviation (e.g., population standard deviation) of pixel intensities can be calculated from the image histogram. The inventors discovered that the degree of motion is positively correlated to the standard deviation of pixel intensity values. Accordingly, a higher standard deviation of pixel intensity values correlates to higher motion artifacts in the analyzed DSA image(s), while a lower standard deviation of pixel intensity values correlates to lower motion artifacts in the analyzed DSA image(s).

This relationship offers a quantifiable approach for assessing the degree of motion in each set of acquired images. Conventionally, practitioners determine the frame rate arbitrarily using only qualitative, subjective insights. In contrast, determining the degree of motion using an objective approach such as disclosed herein enables corresponding frame rate determinations to be made in an objective, quantitative manner.

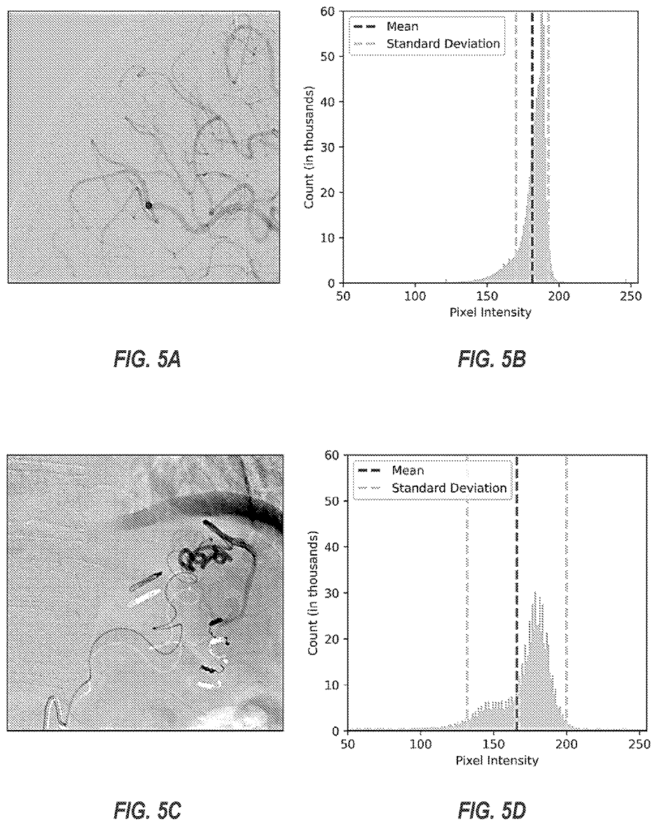

FIGS. 5A-5D illustrate representative examples of DSA images and corresponding pixel intensity histograms. FIG. 5A is an example of a DSA image with a relatively low level of motion artifacts, and FIG. 5B is its corresponding histogram. FIG. 5C is an example of a DSA image with a relatively high level of motion artifacts, and FIG. 5D is its corresponding histogram. As shown, the standard deviation of pixel intensities for the image of FIGS. 5A and 5B is relatively small, whereas the standard deviation of pixel intensities for the image of FIGS. 5C and 5D is relatively large. In other words, the standard deviation of pixel intensities is positively correlated with the degree of motion artifacts in the DSA images.

Other embodiments can additionally or alternatively utilize a machine learning algorithm, a deep-learning approach, and/or a computer vision approach (e.g., one that incorporates feature matching techniques) to determine the degree of motion for a set of DSA images. For example, determining the degree of motion can comprise utilizing a machine learning algorithm. The machine learning algorithm can be generated using a machine learning process in which DSA images pre-categorized according to degree of motion are used as a training dataset. Any machine learning and/or regression technique suitable for regression and classification tasks can be utilized. Examples include gradient boosting, logistic regression, support vector machines (SVM), random forest models, k-nearest neighbors (k-NN) and the like.

### WORKING EXAMPLES

### Example 1: Standard Deviation of Pixel Intensities

DSAs of the cerebral, hepatic, and splenic vasculature were retrospectively collected (n=88 patients, 217 series; frame rate range 1-7 frames per second). The degree of motion artifacts in each DSA was visually scored using a five-grade Likert scale. In addition, image histograms were calculated for each DSA, and the relationship between the standard deviation of pixel intensities and the artifact rating was investigated. A Pearson correlation coefficient with a two-sided p-value was used to quantify the relationship, and the processing time required to analyze each DSA image was recorded.

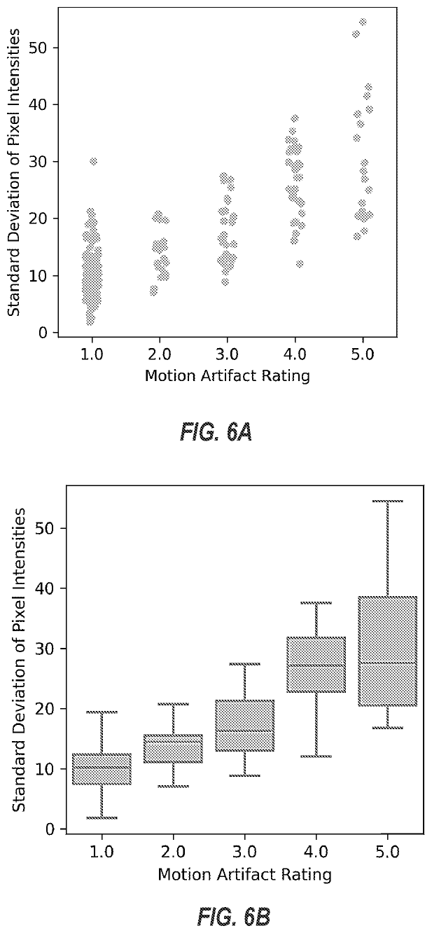

FIGS. 6A and 6B illustrate boxplots and categorical scatterplots, respectively, showing a strong association between the presence of motion artifacts and the standard deviation of pixel intensities. The degree of motion artifacts was strongly associated with the standard deviation of pixel intensities in the image histogram (r=0.78, p<1×10⁻⁵). The average processing time required to calculate this metric for a DSA image was 0.008 seconds on a middle-entry PC (Intel Core i5 processor, 8 GB RAM).

The standard deviation of pixel intensities in DSA is significantly associated with the degree of motion artifacts and can be calculated in less than 0.01 seconds. This relationship can be utilized to adjust acquisition frame rates for DSA in a quantifiable, automated, real-time approach.

### Example 2: Gradient Boosting Machine Learning

A single institution dataset consisting of 88 patients aged 18 years or older was collected with institutional review board approval. From these patients, 217 DSA series of the cerebral, hepatic, and visceral vasculature were selected. The degree of motion artifacts present in each series was manually visually rated on a scale from 1 (minimal motion) to 5 (maximal motion). The data was then randomly split at the patient level into training and testing datasets using an 80-20 percent split.

A gradient boosting machine learning algorithm (XGBoost, v1.6.1) was selected. This algorithm was optimized using the training dataset and evaluated on the testing dataset.

Algorithm performance was assessed using a Pearson correlation coefficient between the predicted and ground truth motion artifact ratings. Statistical significance was assessed using a two-sided p-value.

The degree of motion artifacts present was reliably assessed using a machine-learning based approach, with a strong and statistically significant linear relationship between predicted and ground truth motion artifact ratings (r=0.92, p<1×10⁻⁵). Moreover, this assessment was made in real time, with an average processing time of 0.011 seconds per image.

### Computer System Details

Embodiments described herein can include computer systems. The disclosed computer systems can include one or more processors and computer-readable media such as computer memory stored on one or more hardware storage devices. The computer memory may store computer-executable instructions that when executed by one or more processors cause various functions to be performed, such as the acts recited herein.

Computer-readable media can include any media that can be accessed by the controller. Physical computer-readable storage media includes RAM, ROM, EEPROM, optical or magnetic storage devices, or any other medium which can be used to store desired program code means in the form of computer-executable instructions or data structures and which can be accessed by a computer.

Computer functionality can additionally or alternatively be carried out by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), and the like.

The computer system may be interconnected to one or more other computer systems via one or more network connections. Network connections may include, but are not limited to, connections via wired or wireless Ethernet, cellular connections, or even computer to computer connections through serial, parallel, USB, or other connections. The computer system may be included in a distributed system environment in which local and remote computer systems, which are linked (either by hardwired data links, wireless data links, or by a combination of hardwired and wireless data links) through a network, both perform tasks. In a distributed system environment, program modules may be located in both local and remote memory storage devices.

### EXAMPLE EMBODIMENTS

The following clauses represent a non-exhaustive list of example embodiments of the present disclosure:
Clause 1. A method of generating digital subtraction angiography (DSA) images using a dynamic frame rate, the method comprising: selecting an initial imaging frame rate for X-ray imaging of target anatomy; obtaining a mask image of the target anatomy; obtaining a first set of images at the initial imaging frame rate; generating a first set of DSA images from the first set of images via digital subtraction of the mask image; determining a degree of motion in the first set of DSA images; adjusting the initial imaging frame rate to a first adjusted frame rate based on the determined degree of motion in the first set of DSA images; obtaining a second set of images at the first adjusted frame rate; and generating a second set of DSA images from the second set of images via digital subtraction of the mask image.
Clause 2. The method of clause 1, wherein adjusting the initial imaging frame rate comprises overlaying a degree of motion scale and a frame rate scale and adjusting the initial imaging frame rate such that the first adjusted frame rate is closer to a frame rate corresponding to the determined degree of motion.
Clause 3. The method of any preceding clause, further comprising: determining a degree of motion in the second set of DSA images; adjusting the frame rate to a second adjusted frame rate based on the determined degree of motion in the second set of DSA images; obtaining a third set of images at the second adjusted frame rate; and generating a third set of DSA images from the third set of images via digital subtraction of the mask image.
Clause 4. The method of any preceding clause, further comprising determining a degree of motion for each of multiple successive sets of DSA images and adjusting the frame rate for each subsequent set of DSA images based on the most recent degree of motion measured.
Clause 5. The method of any preceding clause, wherein the initial imaging frame rate is selected based on the target anatomy.
Clause 6. The method of clause 5, wherein the initial imaging frame rate is 1-3 frames per second (fps) for cerebral angiography, 4-12 fps for visceral angiography, or 15-30 fps for pulmonary angiography.
Clause 7. The method of any preceding clause, wherein determining the degree of motion in the first set of DSA images comprises: generating an image histogram from at least one DSA image of the first set of DSA images; calculating a standard deviation of pixel intensities from the image histogram; and determining the degree of motion in the first set of DSA images based on the calculated standard deviation of pixel intensities, wherein the degree of motion is positively correlated to the standard deviation of pixel intensities.
Clause 8. The method of any preceding clause, wherein the degree of motion in the first set of DSA images comprises a Likert scale or numerical scale.
Clause 9. The method of any preceding clause, wherein determining the degree of motion in the first set of DSA images comprises utilizing a machine learning algorithm, wherein the machine learning algorithm is generated using a machine learning process in which DSA images pre-categorized according to degree of motion are used as a training dataset.
Clause 10. The method of clause 9, wherein the machine learning process comprises a gradient boosting classifier.
Clause 11. The method of any preceding clause, wherein determining the degree of motion in the first set of DSA images comprises use of a computer vision approach.
Clause 12. The method of any preceding clause, wherein determining the degree of motion in the first set of DSA images comprises use of a deep-learning approach.
Clause 13. A system for generating digital subtraction angiography (DSA) images using a dynamic frame rate, the system comprising one or more processors and one or more hardware storage devices, the one or more hardware storage devices comprising instructions stored thereon which are executable by the one or more processors to cause the system to: receive input selecting an initial imaging frame rate for X-ray imaging of target anatomy; instruct an X-ray device to obtain a mask image of the target anatomy; instruct the X-ray device to obtain a first set of images at the initial imaging frame rate; generate a first set of DSA images from the first set of images via digital subtraction of the mask image; determine a degree of motion in the first set of DSA images; adjust the initial imaging frame rate to a first adjusted frame rate based on the determined degree of motion in the first set of DSA images; instruct the X-ray device to obtain a second set of images at the first adjusted frame rate; and generate a second set of DSA images from the second set of images via digital subtraction of the mask image.
Clause 14. The system of clause 13, wherein the system is configured to increase the frame rate when the determined degree of motion of the first set of DSA images surpasses a predetermined threshold and to decrease the frame rate when the determined degree of motion of the first set of DSA images is below a predetermined threshold.
Clause 15. The system of clause 13 or clause 14, wherein the system is further configured to: determine a degree of motion in the second set of DSA images; adjust the frame rate to a second adjusted frame rate based on the determined degree of motion in the second set of DSA images; obtain a third set of images at the second adjusted frame rate; and generate a third set of DSA images from the third set of images via digital subtraction of the mask image.
Clause 16. The system of any one of clauses 13-15, wherein the system is configured to determine a degree of motion of multiple sets of DSA images and to adjust the frame rate for a subsequent set of DSA images based on the most recent degree of motion measured.
Clause 17. The system of any one of clauses 13-16, wherein the system is further configured to: generate an image histogram for at least one DSA image of the first set of DSA images; calculate a standard deviation of pixel intensities from the image histogram; and determine the degree of motion in the first set of DSA images based on the calculated standard deviation of pixel intensities, wherein the degree of motion is positively correlated to the standard deviation of pixel intensities.
Clause 18. The system of clause 16, wherein the degree of motion in the first set of DSA images comprises a Likert scale or numerical scale positively correlated to the standard deviation of pixel intensities.
Clause 19. The system of any one of clauses 13-18, wherein the system is configured to receive input selecting a target anatomy and to select the initial imaging frame rate according to the selected target anatomy.
Clause 20. The system of any one of clauses 13-19, wherein the system determines the degree of motion in the first set of DSA images utilizing a machine learning algorithm, wherein the machine learning algorithm is generated using a machine learning process in which DSA images categorized according to degree of motion are used as a training dataset.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

In addition, unless otherwise indicated, numbers expressing quantities, constituents, distances, or other measurements used in the specification and claims are to be understood as optionally being modified by the term “about.” When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, image acquisition and/or analysis steps that are not specifically disclosed herein may optionally be expressly omitted from the disclosed embodiments.

1. A method of generating digital subtraction angiography (DSA) images using a dynamic frame rate, the method comprising:
selecting an initial imaging frame rate for X-ray imaging of target anatomy;
obtaining a mask image of the target anatomy;
obtaining a first set of images at the initial imaging frame rate;
generating a first set of DSA images from the first set of images via digital subtraction of the mask image;
determining a degree of motion in the first set of DSA images;
adjusting the initial imaging frame rate to a first adjusted frame rate based on the determined degree of motion in the first set of DSA images;
obtaining a second set of images at the first adjusted frame rate; and
generating a second set of DSA images from the second set of images via digital subtraction of the mask image.

2. The method of claim 1, wherein adjusting the initial imaging frame rate comprises overlaying a degree of motion scale and a frame rate scale and adjusting the initial imaging frame rate such that the first adjusted frame rate is closer to a frame rate corresponding to the determined degree of motion.

3. The method of claim 1, further comprising:
determining a degree of motion in the second set of DSA images;
adjusting the frame rate to a second adjusted frame rate based on the determined degree of motion in the second set of DSA images;
obtaining a third set of images at the second adjusted frame rate; and
generating a third set of DSA images from the third set of images via digital subtraction of the mask image.

4. The method of claim 1, further comprising determining a degree of motion for multiple successive sets of DSA images and adjusting the frame rate for each subsequent set of DSA images based on the most recent degree of motion measured.

5. The method of claim 1, wherein the initial imaging frame rate is selected based on the target anatomy.

6. The method of claim 5, wherein the initial imaging frame rate is 1-3 frames per second (fps) for cerebral angiography, 4-12 fps for visceral angiography, or 15-30 fps for pulmonary angiography.

7. The method of claim 1, wherein determining the degree of motion in the first set of DSA images comprises:
generating an image histogram from at least one DSA image of the first set of DSA images;
calculating a standard deviation of pixel intensities from the image histogram; and
determining the degree of motion in the first set of DSA images based on the calculated standard deviation of pixel intensities, wherein the degree of motion is positively correlated to the standard deviation of pixel intensities.

8. The method of claim 1, wherein the degree of motion in the first set of DSA images comprises a Likert scale or numerical scale.

9. The method of claim 1, wherein determining the degree of motion in the first set of DSA images comprises utilizing a machine learning algorithm, wherein the machine learning algorithm is generated using a machine learning process in which DSA images pre-categorized according to degree of motion are used as a training dataset.

10. The method of claim 9, wherein the machine learning process comprises a gradient boosting classifier.

11. The method of claim 1, wherein determining the degree of motion in the first set of DSA images comprises use of a computer vision approach.

12. The method of claim 1, wherein determining the degree of motion in the first set of DSA images comprises use of a deep-learning approach.

13. A system for generating digital subtraction angiography (DSA) images using a dynamic frame rate, the system comprising one or more processors and one or more hardware storage devices, the one or more hardware storage devices comprising instructions stored thereon which are executable by the one or more processors to cause the system to:
receive input selecting an initial imaging frame rate for X-ray imaging of target anatomy;
instruct an X-ray device to obtain a mask image of the target anatomy;
instruct the X-ray device to obtain a first set of images at the initial imaging frame rate;
generate a first set of DSA images from the first set of images via digital subtraction of the mask image;
determine a degree of motion in the first set of DSA images;
adjust the initial imaging frame rate to a first adjusted frame rate based on the determined degree of motion in the first set of DSA images;
instruct the X-ray device to obtain a second set of images at the first adjusted frame rate; and
generate a second set of DSA images from the second set of images via digital subtraction of the mask image.

14. The system of claim 13, wherein the system is configured to increase the frame rate when the determined degree of motion of the first set of DSA images surpasses a predetermined threshold and to decrease the frame rate when the determined degree of motion of the first set of DSA images is below a predetermined threshold.

15. The system of claim 13, wherein the system is further configured to:
determine a degree of motion in the second set of DSA images;
adjust the frame rate to a second adjusted frame rate based on the determined degree of motion in the second set of DSA images;
obtain a third set of images at the second adjusted frame rate; and
generate a third set of DSA images from the third set of images via digital subtraction of the mask image.

16. The system of claim 13, wherein the system is configured to determine a degree of motion of multiple sets of DSA images and to adjust the frame rate for a subsequent set of DSA images based on the most recent degree of motion measured.

17. The system of claim 13, wherein the system is further configured to:
generate an image histogram for at least one DSA image of the first set of DSA images;
calculate a standard deviation of pixel intensities from the image histogram; and
determine the degree of motion in the first set of DSA images based on the calculated standard deviation of pixel intensities, wherein the degree of motion is positively correlated to the standard deviation of pixel intensities.

18. The system of claim 16, wherein the degree of motion in the first set of DSA images comprises a Likert scale or numerical scale positively correlated to the standard deviation of pixel intensities.

19. The system of claim 13, wherein the system is configured to receive input selecting a target anatomy and to select the initial imaging frame rate according to the selected target anatomy.

20. The system of claim 13, wherein the system determines the degree of motion in the first set of DSA images utilizing a machine learning algorithm, wherein the machine learning algorithm is generated using a machine learning process in which DSA images categorized according to degree of motion are used as a training dataset.
